(19) United States
(12) Patent Application Publication
(10) Pub. No.: US 2013/0202577 A1
(43) Pub. Date: Aug. 8, 2013

(54) DIAGNOSTIC AND PROGNOSTIC ASSAY FOR BREAST CANCER

(71) Applicants: Tony Tiganis, Mount Waverley (AU); Benjamin James Shields, Ormond (AU); Catriona Ann McLean, Hawthorn (AU); Robert Sutherland, Lindfield (AU)

(72) Inventors: Tony Tiganis, Mount Waverley (AU); Benjamin James Shields, Ormond (AU); Catriona Ann McLean, Hawthorn (AU); Robert Sutherland, Lindfield (AU)

(73) Assignee: MONASH UNIVERSITY, Clayton (AU)

(21) Appl. No.: 13/576,541

(22) PCT Filed: Feb. 3, 2011
(86) PCT No.: PCT/AU2011/000101

§ 371 Date: Oct. 8, 2012

(30) Foreign Application Priority Data
Feb. 3, 2010 (AU) 2010900414

(51) Int. Cl. G01N 33/68

(52) U.S. Cl. CPC G01N 33/6893
USPC 424/94.6; 435/7.1; 435/196; 530/389.7; 435/6.14; 514/44 R

(57) ABSTRACT

The invention relates to the finding that T cell protein tyrosine phosphatase (TCPTP) has a tumour suppressor function. In one particular application, the invention provides a method of diagnosis and/or prognosis of cancer in a subject involving the assessment, in a suitable body sample, of any lack or reduction of TCPTP protein and/or Ptpn2 gene expression. The invention also provides a method of treating or preventing cancer in a subject involving administering to a subject an agent for modulating the activity of TCPTP.

### FIELD OF THE INVENTION

The invention relates to the finding that T cell protein tyrosine phosphatase (TCPTP) has a tumour suppressor function. In one particular application, the invention provides a method of diagnosis and/or prognosis of cancer in a subject involving the assessment, in a suitable body sample, of any lack or reduction of TCPTP protein and/or Ptpn2 gene expression.

### INCORPORATION BY REFERENCE

This patent application claims priority from:
Australian Provisional Patent Application No 2010900414 titled “Diagnostic and prognostic assay for breast cancer” filed 3 February 2010.

The entire content of this application is hereby incorporated by reference.

### BACKGROUND TO THE INVENTION

Breast cancer is the most frequent malignancy among women, with an estimated one million new cases per year worldwide. In Australia alone, a total of 2,641 women died from breast cancer in 2004.

Breast Cancer Development

The adult mammary epithelium is organised into ducts and lobules. Hyperplasia within the duct and lobules and the generation of abnormal cell layers is the earliest stage in breast cancer and is thought to be a precursor for the development of carcinoma in situ, most commonly ductal carcinoma in situ (DCIS), a non invasive lesion with abnormal cells. DCIS can progress to malignant invasive ductal carcinomas that ultimately account for 60-80% of all breast tumours. Once invasive disease has developed, metastasis is likely with the primary metastatic site being the lymph nodes. The transformation of the breast epithelium to malignant and metastatic disease involves an amalgam of epigenetic and genetic events and is deeply influenced by both oestrogen receptor (ER) and growth factor signalling, in particular that mediated by the epidermal growth factor receptor (EGFR) family of protein tyrosine kinases (PTKs). Approximately 60-70% of breast cancers are ER positive (and respond to anti-oestrogens such as tamoxifen) and are reliant on ER signalling for proliferation and survival. More advanced, aggressive breast tumours are often ER negative and overexpress the EGFR/ErbB1 and ErbB2-4 members of the EGFR family of PTKs, while a smaller proportion (10-17%) express ErbB1 but not ER or ErbB2 (otherwise known as HER2/neu) or the progesterone receptor (PR) and constitute the so-called “triple-negative tumours”.

PTKs and Breast Cancer

Since the discovery of c-src as the first proto-oncogene, numerous PTK pathways have been causally linked to the tumorigenic process. In particular, elevated Src family PTKs (SFK), EGFR and signal transducer and activator-3 (STAT3) signalling has been linked to the promotion of tumour cell growth, proliferation, survival, motility and invasion, the promotion of angiogenesis and the development of chemotherapeutic resistance in a wide variety of human tumours including those of the breast^{1,2}.

In breast cancer, ErbB2 is amplified and overexpressed in 20-30% of primary breast cancers and plays a causal role in mammary carcinogenesis. Indeed, ErbB2 overexpression is an adverse prognostic indicator in early stage disease correlating with the development of high grade tumour and increased nodal metastases³. Also, it has been observed that the expression of ErbB2 is sufficient to transform cells in vitro⁴ , while expression of activated ErbB2 in the mouse mammary gland (under the control of the mouse mammary tumour virus (MMTV) promoter) is known to cause glandular hyperplasia followed by the development of adenocarcinoma⁵. Further, in breast cancer, ErbB2 promotes proliferation and the breakdown of mammary epithelial cell-cell interactions by activating key signalling pathways that include the mitogen-activated protein kinase (MAPK) and phosphatidylinositol 3-kinase (PI3K) cascades and STAT3, leading to loss of polarity and the initiation of invasion⁶.

Other EGFR family PTKs have also been implicated in the development of breast cancer. In particular, ErbB1 is highly expressed in triple-negative tumours and correlates with poor prognosis^{7,8}. Also, although ErbB1 is less transforming than ErbB2⁹, ErbB1 is known to cooperate with c-Src to promote the migration of breast cancer cell lines and anchorage-independent growth and aberrant human mammary epithelial cell acinar formation in 3D cultures^{10,11}. Indeed, elevated Src protein levels and/or activity occur in a striking ˜70% of primary breast cancers and often coincide with

ErbB1 or ErbB2 overexpression^{12,13} and, in DCIS, activated Src correlates with high tumour grade, high proliferation and lower recurrence-free survival¹⁴. Further, SFKs can interact with ErbB2 in the promotion of tumorigenicity and metastases^{15,16}, and may also play an integral role in mediating ER signalling by promoting the activation of MAPK, PI3K and STAT3 signalling pathways and the inducton of c-Myc¹². SFKs also cooperate with ErbB1 to promote breast cancer tumorigenicityl^{10,11,17} and, additionally, activated SFKs and STAT3 may contribute to the development of chemotherapeutic resistance by promoting the expression of c-Myc, cyclin DI and anti-apoptotic genes such as the survivin gene^{13,18}.

STAT3 is constitutively activated in many human breast cancers^{13,19}. In addition to serving as a point of convergence for oncogenic ErbB1/2 and SFKs^{2,20}, recent studies have independently linked elevated STAT3 signalling to breast cancer development. STAT3 is phosphorylated by JAK (Janus activated kinases) PTKs downstream of the common interleukin-6 (IL-6) cytokine family receptor β subunit gp130. Indeed, gene deletion studies in mice indicate that STAT3 is essential for gp130 signalling²¹. In breast cancer, an increase in IL-6/gp130 signalling correlates with poor prognosis²² and promotes an invasive phenotype in mammospheres in vitro²³ , whereas knockdown of STAT3 attenuates xenograft growth and sensitises tumours to chemotherapeutics^{24,25}.

PTPs and Cancer

Protein tyrosine phosphatases (PTPs) are a large and structurally diverse family of enzymes of approximately 100 members typified by the prototypic PTP1B²⁶. In contrast to PTKs, it has only recently become apparent that mutations and altered PTP expression may contribute to tumorigenesis. For example, activating somatic mutations in SHP-2 that promote Ras/MAPK signalling contribute to the onset/progression of several sporadic human malignancies (including ˜35% of juvenile myelomonocytic leukemias)²⁷ whereas PTP1B is overexpressed in 40% of human breast cancers coinciding with ErbB2 amplification and activates the oncoprotein c-Src²⁸. Elegant studies from two independent laboratories have shown that the deletion of PTP1B in MMTV-NDL2 mice (i.e. which express activated murine ErbB2 in mammary tissue), or inhibition of PTP1B activity with a specific PTP1B inhibitor, attenuates mammary tumorigenesis and protects from the development of lung metastases, whereas PTP1B overexpression promotes the development of spontaneous breast cancer, thus identifying PTP as a bona fide oncoprotein²⁹⁻³¹. However, in most cases, PTPs may act as tumour suppressors and inhibitory mutations, or the suppression of PTP expression may alleviate constraints on tyrosine phosphorylation-dependent signalling. Thus, the loss or inactivation of a PTP may act in concert with oncogenic activated or overexpressed PTKs to promote tumorigenesis. In this regard, it is notable that in the last few years, numerous PTPs have been identified as potential tumour suppressors including PTPRJ (DEP-1), which is deleted or mutated in human colon (˜39%), lung and breast cancers^{32,33}, PTPRO whose expression is diminished in various human cancers as a result of CpG island promoter methylation³⁴ and PTPRT/PTPρ that is mutated in human colorectal cancer³⁵.

TCPTP

T cell protein tyrosine phosphatase (TCPTP), also known as tyrosine-protein phosphatase non-receptor type 2 (PTPN2), is a ubiquitous tyrosine-specific phosphatase that is expressed as two splice variants: a 48 kDa form (TC48) that, like PTP1B, is targeted to the endoplasmic reticulum, and a 45 kDa variant (TC45) that is targeted to the nucleus by a nuclear localisation sequence. Despite an apparent exclusive nuclear localisation in resting cells, TC45 can exit the nucleus in response to varied stimuli and therefore access substrates both in the cytoplasm and nucleus^{36,37}. That is, in the cytoplasm, TC45 interacts with substrates including receptor PTKs such as the IR 62 and ErbB1 and non-receptor PTKs such as c-Src (nb TCPTP dephophorylates the Y418 autophosphorylation site) and JAK1/3³⁸, whereas TC45's nuclear substrates include STAT family members such as STAT-1, 3, 5 and 6³⁹⁻⁴¹. Further, TC45's spatial isolation in the nucleus may be essential for the initiation of signal transduction at the cell surface and its nuclear exit may represent a negative feedback loop for the coordinated suppression of signalling.

TCPTP and Tumorigenesis

Previously, the present applicant has reported that TCPTP suppresses the activation of ErbB1 in response to ligand or integrin transactivation and dephosphorylates and suppresses the tumorigenicity associated with a constitutively active ErbB1 mutant known as AEGFR in vitro and in U87MG glioblastoma cell (intracranial) xenografts⁴². Recently, Matilla et al. (2005)⁴³ demonstrated that in response to integrin ligation, TCPTP specifically interacts with the collagen binding α₁β₁ integrin to suppress ErbB1 activation and the associated tumorigenicity in HeLa cervical adenocarcinoma cells. In more recent studies by the applicant, it has been shown that TCPTP serves as an integral regulator of DNA replication checkpoint response, arguably the most important checkpoint for preventing the genetic instability associated with cancer. In particular, these studies have shown that TC45 inactivates STAT3 signalling to prevent checkpoint bypass and unscheduled cell division in response to DNA replication stress. In the absence of TCPTP, mouse embryonic fibroblasts (MEFs) progress through mitosis with lagging chromosomes allowing for aneuploidy and the acquisition of a transformed phenotype as monitored by anchorage independence (data not shown). Importantly, it has been shown that TCPTP knockdown in HeLa cells abrogates an otherwise intact DNA replication checkpoint response, whereas TC45 reconstitution into U87MG glioblastoma cells, which express little or no TC45 and have a defective checkpoint, reinstates the checkpoint. Although collectively these results indicate that TCPTP has the capacity to regulate oncogenic PTK pathways that contribute to the genesis and development of human tumours, it has heretobefore remained unclear as to whether TCPTP may serve as a tumour suppressor. Indeed, while polymorphisms in Ptpn2 have been linked with the development of several inflammatory disorders⁴⁴, no similar SNPs or mutations have been identified in human tumours. Nevertheless, the applicant has found that the acquisition of resistance to STI571 in CML cells in vitro is accompanied by the down-regulation of TCPTP, whereas increased TCPTP expression in activated-B-cell-like v/s germinal centre B-cell-like diffuse large B cell lymphomas may suppress STAT6 signalling and contribute to the differential biological characteristics of these tumour subtypes.

On the basis of these findings, together with the observation that ErbB1, SFKs and STAT3 can serve as bona fide substrates for TCPTP, the realisation that TCPTP attenuates the tumorigenicity that is associated with the overexpression/activation of ErbB1 and/or SFKs in tumour cells and the genomic instability that can be associated with PTK hyperactivation⁴², and the discovery (described hereinafter) that TCPTP expression is reduced in a large number of breast cancer cell lines and appears to contribute to their tumorigenicity, indicates that TCPTP is, indeed, a tumour suppressor in, at least, breast cancer. As a consequence, the present applicant hereby proposes the use of TCPTP, or “TCPTP status”, as the basis of a diagnostic and/or prognostic assay for breast cancer in a subject.

### SUMMARY OF THE INVENTION

The present invention provides, in a first aspect, a method of diagnosis and/or prognosis of cancer in a subject, said method comprising the step of assessing, in a suitable body sample from said subject, any lack or reduction of TCPTP protein and/or Ptpn2 gene expression.

In a second aspect, the present invention provides a method for assessing a subject's predisposition to cancer, said method comprising the step of assessing, in a suitable body sample from said subject, any lack or reduction of TCPTP protein and/or Ptpn2 gene expression.

In a third aspect, the present invention provides a method for assisting the selection of a therapy for a cancer in a subject, said method comprising the step of assessing, in a suitable body sample from said subject, any lack or reduction of TCPTP protein and/or Ptpn2 gene expression.

In a fourth aspect, the present invention provides a method of treating or preventing cancer in a subject, wherein said method comprises administering to said subject an agent for modulating the activity of TCPTP, optionally in combination with a pharmaceutically acceptable carrier and/or excipient.

In further aspects, the present invention provides the use of an agent for modulating the activity of TCPTP, optionally in combination with a pharmaceutically acceptable carrier and/or excipient, for the treatment or prevention of cancer; the use of an agent for modulating the activity of TCPTP in the preparation of a pharmaceutical composition for treating or preventing cancer; and a pharmaceutical composition for treating or preventing cancer, wherein said composition comprises an agent for modulating the activity of TCPTP in combination with a pharmaceutically acceptable carrier and/or excipient.

### BRIEF DESCRIPTION OF THE ACCOMPANYING FIGURES

FIG. 1 shows the results of immunoblots conducted on a number of breast cancer cell lines for expression of TCPTP 45 and 48 kDa variants; cell lines showing a deficiency of TC45 are highlighted with *, cell lines showing a deficiency of both TC45 and TC48 are indicated by **;

FIG. 2 provides images of immunoblots showing enhanced EGF-induced (A) and integrin-induced (B) signalling in TCPTP-deficient HeLa cells;

FIG. 3 provides images and graphical results showing the effect of TCPTP-deficiency in HeLa cells (A), ER⁺ T47D mammary ductal carcinoma cells (B), and reconstituted TC45 in MDA-MB-231 ErbB1⁺ mammary adenocarcinoma cells (C) on anchorage-independent growth of the cells in soft agar;

FIG. 4 provides graphical results showing (A) that TC45 suppresses the growth of MDA-MB-231 xenografts in nude mice, but in TCPTP knockdown HeLa cells, TCPTP-deficiency enhances the growth of HeLa xenografts (B);

FIG. 5 provides images of MCF-7 cell (which have low TC45 expression) colonies showing that “reconstitution” of TC45 through expression from a retrovirus (pwzl-TC45) inhibits MCF-7 cell growth and brings about cell death;

FIG. 6 provides images showing immunoreactivity of the specific TCPTP monoclonal antibody (CF4) in (A) TCPTP-deficient and TCPTP-expressing HeLa tumour xenografts, (B) human breast cancer tissue homogenates and (C) formalin fixed and paraffin imbedded sections; and

FIG. 7 provides graphical results for survival and relapse for quartiles of 243 breast cancer patients based on Ptpn2 gene expression. (A) shows that the lowest quartile (“1”) of Ptpn2 expressors had a significantly shorter survival time relative to the other quartiles (hazard ratio (HR)+2.0, 95% confidence interval (CI)=1.2-3.3, p=0.009 by univariate Cox regression analysis)(Cum Survival shown as 0-100%; Survival Time shown as time in months). (B) shows that the lowest quartile of Ptpn2 expressors also had a poorer level of relapse-free survival than the other quartiles (HR+1.5, 95% CI+1.0-2.3, p=0.071))(Cum Survival shown as 0-100%; Relapse Time shown as time in months).

### DETAILED DESCRIPTION OF THE INVENTION

The present invention provides, in a first aspect, a method of diagnosis and/or prognosis of cancer in a subject, said method comprising the step of assessing, in a suitable body sample from said subject, any lack or reduction of TCPTP protein and/or Ptpn2 gene expression.

It is considered that the tumour suppressor function of TCPTP is a general tumour suppression function and, as such, the method of the first aspect is expected to be broadly applicable to the diagnosis and/or prognosis of cancer. Nevertheless, it is recognised that the method may be particularly suitable for the diagnosis and/or prognosis of a cancer selected from breast cancers, lung cancers, colon cancers, haematological cancers (e.g. B cell and T cell leukaemias), gliomas and solid tumours generally. It is also recognised that the method of the first aspect is particularly suitable for the prognosis and/or diagnosis of a breast cancer selected from the group consisting of those that are ER positive ErbB2 negative (ER⁺ErbB2⁻), ER negative ErbB2 positive (ER⁻ErbB2⁺) and ER negative ErbB2 negative (ER⁻ErbB2⁻), and a breast cancer that is a triple-negative tumour (ER negative ErbB2 negative PR negative; ER⁻ErbB2⁻PR⁻).

Preferably, the method is applied to the prognosis of cancer such as a breast cancer, wherein the detection of a lack or reduction of TCPTP protein and/or Ptpn2 gene expression may provide information on, or identify, one or more of the cancer stage, rate of cancer progression and cancer genetics/epigenetics which, in turn, enables a prognosis on the future course and/or outcome of the disease in the said subject. For example, in the context of a triple-negative tumour, the detection of a lack or reduction of TCPTP protein or Ptpn2 gene expression predicts a worse subject outcome (e.g. with or without suitable therapeutic intervention), whereas if the sample shows no apparent lack or reduction of TCPTP protein and/or Ptpn2 gene expression (i.e. there are normal levels of TCPTP protein and/or Ptpn2 gene expression) than the prognosis may be better. Of course, the levels of TCPTP protein and/or Ptpn2 gene expression may change with cancer progression and, as such, it may be preferable to repeat the step of assessing a lack or reduction of TCPTP protein and/or Ptpn2 gene expression at one or more time points.

Thus, in an embodiment, the method of the first aspect comprises:

(i) a first step of assessing, in a first suitable body sample from said subject, any lack or reduction of TCPTP protein and/or Ptpn2 gene expression; and

(ii) one or more further step(s) of assessing any lack or reduction of TCPTP protein and/or Ptpn2 gene expression, in a body sample(s) that is substantially equivalent to the first body sample but taken from the subject at one or more later time point(s).

As such, the method may employ serial steps of assessing the level of TCPTP protein and/or Ptpn2 gene expression, and wherein it becomes apparent that there has been a reduction, or further reduction (i.e. relative to a previously detected reduced level), in the level of TCPTP protein and/or Ptpn2 gene expression, that reduction is indicative of cancer progression to, for example, a later cancer stage or increased tumorigenicity. In turn, this may predict a worse cancer prognosis (e.g. with or without suitable therapeutic intervention).

The suitable body sample may vary depending upon the nature and type of the cancer, and may be, for example, a sample of blood, serum, urine, or cheek cell swab. However, typically, the body sample will be a sample of tumour tissue (e.g. a tissue biopsy).

The TCPTP protein level in the body sample may be detected by any of the methods well known to the person skilled in the art, for example, immunoassays such as enzyme-linked immunosorbant assay (ELISA), radioimmunoassay (RIA) or immunohistochemistry (e.g. with sectionalised samples of a tissue biopsy) using an anti-TCPTP antibody or fragment thereof (e.g. a polyclonal or monoclonal antibody or fragment thereof such as an Fv, Fab and F(ab)₂ fragment that is capable of binding TCPTP, and recombinant antibodies that bind to TCPTP such as a single chain antibody (e.g. scFV antibodies)) or other ligand that binds to TCPTP such as, for example, peptides, polypeptides, nucleic acids or aptamers (e.g. nucleic acid or peptide aptamers). Particularly suitable methods for determining the level of TCPTP present in a test body sample are immunoassays utilising labelled molecules in various sandwich, competition, or other assay formats which may provide a signal, the strength of which may be correlated to the level of TCPTP present in a sample.

The level of Ptpn2 gene expression in the body sample may be assessed by any of the methods well known to the person skilled in the art, for example, assays for measuring the level of mRNA transcribed from Ptpn2 such as quantitative amplification techniques (e.g. quantitative reverse transcription polymerase chain reaction (RT-PCR)) and probe hybridisation methods (e.g. Northern blotting). Also, the level of Ptpn2 gene expression may be assessed by determining the presence of one or more non-functional allele of Ptpn2. As used herein, the term “non-functional allele” refers to an allele of the Ptpn2 gene that either can not be substantially expressed or, otherwise, encodes an inactive or substantially inactive TCPTP enzyme (as determined by, for example, a standard TCPTP activity assay (e.g. DuoSet® IC Catalog # DYC2468-2 and DYC2468-5; R&D Systems, Inc., Minneapolis, Minn., United States of America)). A heterozygous subject, or heterozygous cells of a sample therefrom, therefore includes one non-functional Ptpn2 allele and one functional Ptpn2 allele (i.e. Ptpn2+/−), while a homozygous subject, or homozygous cells of a sample therefrom, includes either two non-functional Ptpn2 alleles (i.e. Ptpn2−/−) or two functional Ptpn2 alleles (i.e. Ptpn2+/+). It will be understood by the person skilled in the art that functional Ptpn2 alleles may encode, for example, TC48 and/or the truncated variant TC45 form. It will also be understood by the person skilled in the art that a non-functional allele of Ptpn2 gene may be the result of genetic mutation such as, for example, deletion of the allele, truncation of the allele, a missense mutation, a nonsense mutation, a frameshift mutation or a splice-site mutation, or epigenetic changes (e.g. DNA hypomethylation or hypermethylation). The presence or absence of at least one non-functional allele of the Ptpn2 gene may be determined using any of the methods well known to the person skilled in the art. Thus, the step of assessing the presence or absence of at least one non-functional allele of the Ptpn2 gene may comprise probing genomic DNA (e.g. by Southern blotting with appropriate DNA probes) or amplifying the Ptpn2 gene or a chromosomal locus or region normally expected to include the Ptpn2 gene (e.g. a locus or region at human chromosome 18p11) with suitable primer sequences. For amplification reactions, the primer sequences may be designed to amplify all Ptpn2 gene sequences present in the sample, thereby necessitating the determination of the sequences of the amplified DNA (i.e. to determine whether the amplified sequences represent a non-functional and/or functional Ptpn2 gene alleles), or may otherwise be designed to amplify, under appropriate conditions, only a Ptpn2 gene sequence representing a non-functional allele (or a portion thereof). In an embodiment, a suitable amplification may be a multiplex reaction utilising two or more pairs of primer sequences designed to amplify two or more known non-functional Ptpn2 gene alleles (or a portion thereof). For the detection of Ptpn2 promoter hypomethylation or hypermethylation, any of the standard assay methods known to the person skilled in the art may be used (e.g. the methylation status of predicted CpG islands can be assessed by methylation-sensitive high resolution melting analysis of bisulphite-treated DNA^{47,48} using a standard commercially available kit (e.g. EpiTect Bisulphite Kit; Qiagen, Germantown, Md., United States of America)); preferably, such methods will involve analysis of one or both of two putative CpG islands in the promoter region of Ptpn2 of 549 and 725 nucleotides in length (72-74% CG) occurring within a 1.36 kb region extending from the promoter (−491 relative to ATG) into intron 1 (+872 relative to ATG).

In the context of cancer prognosis, detecting the presence of at least one non-functional Ptpn2 gene allele (or a reduction of Ptpn2 gene expression) is indicative of a poorer prognosis (without suitable intervention) than for a similar cancer from a subject, or sample thereof, having two functional Ptpn2 gene alleles (i.e. Ptpn2+/+) and normal levels of expression of TCPTP protein. Further, for a subject, or sample thereof, having two non-functional alleles of the Ptpn2 gene (i.e. Ptpn2−/−) and/or a lack of Ptpn2 gene expression, the prognosis of a cancer is expected to be poorer (without suitable intervention) than the prognosis of a similar cancer from a subject, or a sample thereof, having one non-functional Ptpn2 gene allele (i.e. a heterozygous Ptpn2+/− genotype) and/or a reduction of Ptpn2 gene expression.

Preferably, the method of the first aspect comprises assessing TCPTP protein. This may involve detecting the level of total active TCPTP (e.g. TC45+TC48) or, more preferably, just the level of TC45, since the studies described hereinafter indicate that a deficiency in TC45 alone is sufficient to promote/enhance oncogenic PTK signalling and contribute to the tumorigenic process in cancers such as breast cancers. Accordingly, the method may preferably comprise detecting the level of TC45 through the use of a ligand which binds specifically to TC45. As used herein, the term “binds specifically” (and “specifically binds”) means that the ligand should not bind substantially to (that is, substantially “cross-react” with) another peptide, polypeptide or substance present in the test body sample (e.g. TC48 and the closely related phosphatase, PTP1B). Preferably, the ligand binds with at least 3 times higher, more preferably at least 10 times higher, and most preferably at least 50 times higher affinity to TC45 than any other relevant peptide, polypeptide or substance. Preferably, the ligand will therefore be an antibody or fragment thereof which specifically binds TC45.

In some embodiments, the method may further comprise assessing the sample for one or more marker for cancers such as breast cancers (e.g. ER, ErbB1, ErbB2, PR, c-SRC, STAT-3, BRCA1, BRCA2 and IL-6/gp130).

In a second aspect, the present invention provides a method for assessing a subject's predisposition to cancer, said method comprising the step of assessing, in a suitable body sample from said subject, any lack or reduction of TCPTP protein and/or Ptpn2 gene expression.

A subject determined as having, for example, at least one non-functional allele of Ptpn2 gene (i.e. a subject that has a Ptpn2+/− or Ptpn2−/− genotype) is likely to have a greater predisposition to cancers such as breast cancers (particularly, a breast cancer selected from the group consisting of those that are ER⁺ErbB2⁻, ER⁻ErbB2⁺, ER⁻ErbB2⁻ and ER⁻ErbB2PR⁻).

In a third aspect, the present invention provides a method for assisting the selection of a therapy for a cancer in a subject (such as in a “personalised medicine” approach), said method comprising the step of assessing, in a suitable body sample from said subject, any lack or reduction of TCPTP protein and/or Ptpn2 gene expression.

Preferably, the body sample will be a tumour tissue sample.

The method of the third aspect is particularly suitable for assisting in the selection of a chemotherapy for a cancer in a subject by, for example, providing information to identify chemotherapeutic agents that may bring about a desirable therapeutic outcome (e.g. a slowing or diminishing of cancer growth or spread).

In a fourth aspect, the present invention provides a method of treating or preventing cancer in a subject, wherein said method comprises administering to said subject an agent for modulating the activity of TCPTP, optionally in combination with a pharmaceutically acceptable carrier and/or excipient.

Preferably, the agent enhances the activity of TCPTP (i.e. is a TCPTP enhancing agent). Such an agent may provide TCPTP to a subject (or tissue thereof) lacking TCPTP, or may simply increase the amount of endogenous TCPTP present. Such an agent may be selected from TCPTP (preferably TC45) preferably including a native or heterologous nuclear localisation signal (NLS), agents which enhance transcription or translation of the Ptpn2 gene (e.g. a transcription factor associated with Ptpn2 over-expression) and gene therapy agents such as expression vectors or oligonucleotides or other delivery systems (e.g. viral vectors such as retroviral or adenoviral vectors) containing a polynucleotide sequence encoding TCPTP (preferably TC45) preferably including a native or heterologous NLS. One example of a such a TCPTP enhancing agent, comprising a retrovirus containing a polynucleotide sequence encoding TC45, is described in the following Example and has been shown to inhibit cell proliferation and cause cell death in breast cancer cell lines that have low TC45 expression. Other suitable TCPTP enhancing agents may enhance the activity of endogenous TCPTP in the subject. As used herein, the term “TCPTP enhancing agent” is to be understood as including agents which mimic the activity of TCPTP (e.g. functional fragments of TCPTP, peptide mimetics of the active domains of TCPTP, and small organic molecules which mimic TCPTP activity).

TCPTP modulating agents for use in the method may be formulated into any suitable pharmaceutical composition or dosage form (e.g. compositions for oral, buccal, nasal, intramuscular and intravenous administration). Typically, such a composition will be administered to the subject in an amount which is effective to achieve a therapeutic effect, and may therefore provide between about 0.01 and about 100 μg/kg body weight per day of the TCPTP modulating agent, and more preferably, provide from 0.05 and 25 μg/kg body weight per day of the TCPTP modulating agent. A suitable composition may be intended for single daily administration, multiple daily administration, or controlled or sustained release, as needed to achieve the most effective results.

The term “pharmaceutically acceptable” means that the carrier and/or excipient is approved by a regulatory agency of the federal or a state government or listed in the US Pharmacopoeia or other generally recognised pharmacopoeia for use in animals, and, more particularly, in humans. Suitable carriers include: sterile liquids such as water and/or oils, including those of petroleum, animal, vegetable, or synthetic origin, such as peanut oil, soybean oil, mineral oil, sesame oil and the like, as well as saline solutions, blood plasma medium, aqueous dextrose and glycerol solutions, particularly for injectable solutions; and solid materials including pharmaceutical grades of mannitol, lactose, starch, magnesium stearate, sodium saccharine, cellulose, magnesium carbonate and the like. Suitable excipients include, for example, one or more of the following: binders, fillers, disintegrants, lubricants, coatings, sweeteners, flavours, colourings, preservatives, diluents, adjuvants, and/or vehicles. In some instances, excipients collectively may constitute about 5-95% of the total weight (and/or volume) of a particular dosage form.

Preferably, the cancer to be treated or prevented will be selected from breast cancers (particularly, a breast cancer selected from the group consisting of those that are ER⁺ErbB2⁻, ER⁻ErbB2⁺, ER⁻ErbB2⁻ and ER⁻ErbB2⁻PR⁻).

The method of the fourth aspect may be used as a combination therapy wherein the method further comprises administering a chemotherapeutic agent such as a cytoskeletal disrupting agent, mitosis impairing agent, antiangiogenesis agent and/or apoptosis inducing agent (including, specifically, one or more of tamoxifen, zoledronic acid, vincristine, cytochalasin D, paclitaxel, cisplatin and etoposide), and/or involve radiotherapy.

In the methods of the invention, the subject will typically be human, however it is to be understood that the methods are also applicable to non-human subjects such as, for example, livestock (e.g. cattle, sheep and horses), exotic animals (e.g. tigers and elephants and the like) and companion animals (such as dogs and cats).

In a fifth aspect, the present invention provides the use of an agent for modulating the activity of TCPTP, optionally in combination with a pharmaceutically acceptable carrier and/or excipient, for the treatment or prevention of cancer.

In a related, sixth aspect, the present invention provides the use of an agent for modulating the activity of TCPTP in the preparation of a pharmaceutical composition for treating or preventing cancer.

Further, in a seventh aspect, the present invention provides a pharmaceutical composition for treating or preventing cancer, said composition comprising an agent for modulating the activity of TCPTP in combination with a pharmaceutically acceptable carrier and/or excipient.

It is also to be understood that the present invention relates to kits for use in the methods of the first, second or third aspects, wherein said kits may comprise a TCPTP protein or a functional fragment thereof and/or an anti-TCPTP antibody or fragment thereof. Additionally, or alternatively, such kits may comprise oligonucleotide probes for hybridisation assays or oligonucleotide primers for polynucleotide amplification-based assays. The kits may be provided with instructions for use in the methods of the first, second or third aspects.

The invention is hereinafter described by way of the following non-limiting examples.

### EXAMPLES

### Example 1

### Materials and Methods

Protein Analysis

Cells were harvested in modified RIPA lysis buffer (50 mM HEPES, pH: 7.4, 150 mM NaCl, 10% (v/v) glycerol, 1.5 mM MgCl₂, 1 mM EGTA, 1% (v/v) Triton X-100, 1% (w/v) sodium deoxycholate, 0.1% SDS, 1 mM sodium orthovanadate, 100 mM NaF, 2 mM PMSF, 1 μg/ml leupeptin, 1 μg/ml pepstatin A, 1 mM benzamidine) and kept on ice for 30 minutes to facilitate complete lysis. Lysates were then clarified by centrifugation at 16000 rpm at 4° C. for 15 minutes and protein content analysis performed using the Bradford assay (Biorad).

80 μg of each sample (in 1×Laemmli buffer) was then loaded onto 10% polyacrylamide gels and proteins resolved by SDS-PAGE, before transferring the proteins to PVDF and membranes blocked in 5% non-fat milk/TTBS. Membranes were then probed for total phospho-tyrosine (p-tyr) or the phosphorylated and activated forms of EGFR (Y1173), SFKs (Y418) and STAT3 (Y705), then re-probed for total EGFR, c-src, STAT3, TCPTP, tubulin or actin.

TCPTP Knockdown by shRNA

HeLa adenocarcinoma and T47-D breast carcinoma cells were seeded at 1×10⁵ cells per well into 6 well plates the day before infection. Cells were infected with lentiviruses bearing control (MISSION pLKO.1-Puro) or TCPTP-specific (ID 2783) or TC48-specific (ID 2784) shRNAs (Sigma Chemical Company, St Louis, Mo., United States of America) in the presence of 8 μg/mlhexamethidrine bromide. The following day, culture media was replenished and pools of stably transduced cells were selected and then maintained in the presence of either 1 μg/ml (for HeLa) or 2 μg/ml (for T47-D) puromycin. All experiments were performed in the absence of puromycin.

EGF Stimulation

HeLa cells stably expressing control or TCPTP-specific shRNAs were seeded into 6 well plates at a density of 5×10⁵ per well. The next day, cells were serum starved in media containing 0.1% FCS for 6 hr then stimulated with 2 ng/ml EGF for the indicated times. Cells were harvested in RIPA lysis buffer and lysates processed for immunoblot analysis.

Collagen-Integrin Ligation Assay

Six well plates were coated with 50 μl rat-tail collagen (2 mg/ml; Roche, Basel, Switzerland), air dried and sterilised. HeLa cells stably expressing shRNAs were grown to 80-90% confluency and then serum starved for 24 hours in 0.1% FCS/DMEM. Cells were then washed twice with PBS and incubated with 20 mM EDTA/PBS for 30 min at 37° C./5% CO₂. Detached cells were resuspended in 0.25% BSA/DMEM (-phenol red), pelleted and resuspended in 0.25% BSA/DMEM (-phenol red). This was repeated two more times and the cells then resuspended in 0.1%/DMEM (-phenol red), incubated for 30 minutes at 37° C./5% CO₂. Cells (1×10⁶) were then seeded onto collagen coated plates and adherent cells collected at the indicated times in RIPA lysis buffer and processed for immunoblot analysis.

MDA-MB-231 TC45 DOX Xenograft Assay

MDA-MB-231(ATCC Accession No HTB-26) TC45 DOX cells were grown in the presence (+TC45) or absence (−TC45) of 2 μg/ml doxycycline for 3 days prior to processing for xenograft studies. Cells were washed once with PBS, twice with 10 mM EDTA/PBS and then trypsinised for 5 minutes at 37° C./5% CO₂. Cells were resuspended in 10% FCS/DMEM and rested for 20 minutes at 37° C./5% CO₂, then pelleted, washed with PBS, counted and cell numbers adjusted with the appropriate volume of PBS to achieve a density of 2×10⁷cells/ml in PBS and growth factor-reduced Matrigel (BD Biosciences, Franklin Lakes, N.J., United States of America) added to 50% (v/v). 100 μl (1×10⁶ cells) of the cells/Matrigel suspension was injected subcutaneously into the right flanks of female Balb/c nu/nu mice. Doxycycline (1 mg/ml) was added to the drinking water of mice injected with “+TC45” MDA-MB-231 TC45 DOX cells to maintain TC45 expression. Tumours were measured with calipers every 3-4 days and growth curves plotted.

HeLa Xenograft Assay

HeLa cells (1×10⁶) expressing TCPTP (TC45+TC48)-specific shRNA or control HeLa cells (1×10⁶) were resuspended in 50 μl PBS plus 50 μl growth factor-reduced Matrigel (BD Biosciences) and injected subcutaneously into the right flanks of female Balb/c nu/nu mice. Tumours were measured with calipers every 3-4 days and growth curves plotted.

Soft Agar Assay

Cell suspensions (2×10³ HeLa, 2×10³ T47-D and 5×10³ MDA-MB-231 TC45 DOX) in 0.3% agar/10% FBS DMEM were overlayed onto 0.7% agar/10% FBS DMEM underlays in 6 well plates. To maintain expression of TC45 in MDA-MB-231 TC45 DOX cells, doxycycline was added to the overlays every 3 days. Plates were incubated for 3-4 weeks at 37° C./5% CO₂ until such time that colonies were visible. Colonies were stained in situ with 0.1% crystal violet overnight at 37° C./5% CO₂ and images captured and counted by eye.

TCPTP Reconstitution in MCF-7 Cells

Retroviruses encoding human TC45 were generated in the BING replication-incompetent virus packaging cell line as described previously by Klingler-Hoffmann, M. et al., 2001⁴². Briefly, BING were electroporated with either pWZL(Hygro) control or TC45-pWZL(Hygro) retroviral DNA constructs⁴². Virus-containing supernatants were harvested and added to MCF-7 cells (ATCC Accession No HTB-22) and drug-resistant cell colonies selected in medium containing 100 mg/ml hygromycin B (Invitrogen Corporation, Carlsbad, Calif., United States of America). Cells were fixed in 3% paraformaldehyde and stained with Giemsa (Sigma) to visualise cell colonies.

Breast Cancer Tissue Analysis

Snap frozen breast tumour tissue blocks (Victorian Cancer Biobank, Carlton, VIC, Australia) were trimmed to a size of ˜4 mm², excess blood vessels removed and placed in an Eppendorf® tube containing 200 μl cold RIPA lysis buffer. Samples were mechanically homogenised and sonicated (4×5 minute bursts) and lysates clarified by centrifugation at 4° C. (16000 rpm, 30 min). Clarified lysates were re-centrifuged for a further 15 minutes (16000 rpm, 4° C.) and supernatants processed for immunoblot analysis.

Immunohistochemistry (IHC)

PFA fixed and paraffin embedded sections (Victorian Cancer Biobank) were prepared for immunohistochemistry. TCPTP cells were detected in a two-step antigen labelling procedure using TCPTP primary antibody (CF4) and anti-mouse secondary antibody conjugated to peroxidase. Peroxidase activity (brown staining) was detected using DAB substrate-chromagen as per the instructions of the manufacturer (Dako, Glostrup, Denmark).

Correlation of Ptpn2 Expression to Clinical Variables and Patient Outcome

A previously-generated microarray dataset consisting of gene expression data from the tumours of 243 consecutively collected stage I/II breast cancer patients^{49,50} was interrogated to correlate PTPN2 expression to clinical variables and patient outcome. All patients were younger than 53 years old and had tumours that were less than 5 cm in size. 122 of the patients were lymph node-negative, and the remaining 121 patients were lymph node-positive (i.e. cancer cells were present in the lymph nodes). The median follow-up time was 7.0 years. The gene expression data analysis was performed in R software with additional Bioconductor packages (www.r-project.org and www.bioconductor.org). The primary end points for the survival analyses was either relapse-free survival (RFS) or breast cancer-specific survival (BCSS) which was measured from the date of diagnosis to local or systemic relapse or death from breast cancer, or otherwise censored at the time of the last follow-up visit or at non disease-related death. The time to first relapse or disease-specific death was plotted as Kaplan-Meier survival curves. Cox proportional hazards regression was used for univariate analysis of the prognostic impact of PTPN2 expression. For statistical analysis, SPSS (Version 15.0.1; SPSS, Inc, Chicago, Ill., United States of America) software was used.

Results

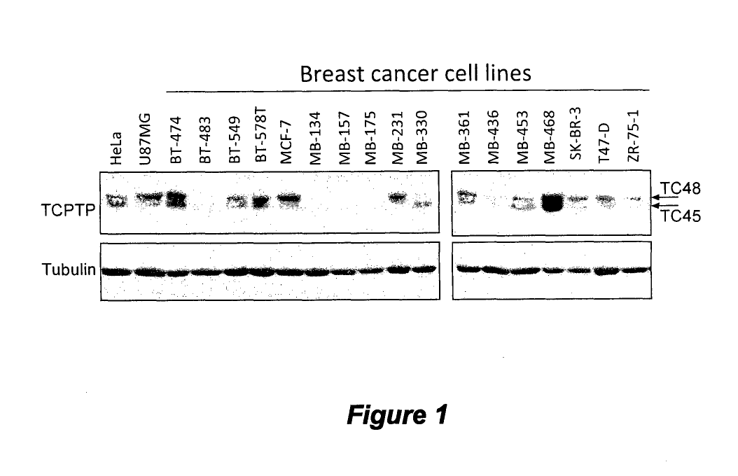

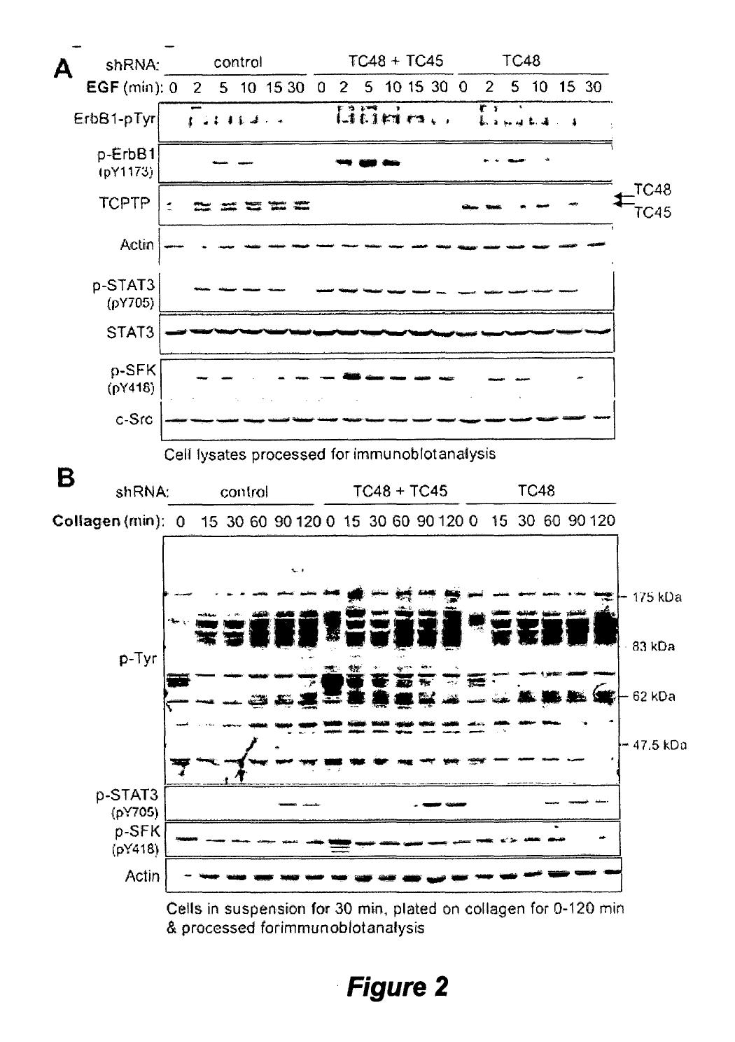

Previously, it was established that changes in TCPTP expression can contribute to the tumorigenic process in cells in vitro^{42,43}. More recently, it was shown that TCPTP-deficiency has the potential to contribute to genomic instability. The studies of this example now indicate that the TCPTP variants may be differentially expressed in human cancer. In particular, in a panel of breast cancer cell lines, either TC45+TC48 or just TC45 protein levels were diminished in about a third of the cell lines tested (see FIG. 1). Further, it was found that a TC45 deficiency on its own in cancer cells may be sufficient to promote oncogenic PTK signalling and tumorigenicity; the results showed that in HeLa adenocarcinoma cells (ATCC Accession No CCL-2), which express similar amounts of TC45 and TC48, shRNA-mediated knockdown (RNAi) of both TC45+TC48, but not TC48 alone, enhanced PTK signalling instigated by growth factors such as EGF (see FIG. 2A) or integrin-ligation (plated on collagen; see FIG. 2B). In addition, the results indicated that TC45, but not TC48, may be essential for the inactivation of SFK signalling when cells are no longer attached to an extracellular matrix; this is a fundamental mechanism that normally serves to prevent the survival and growth of detached cells which, in cancer, is often “derailed” allowing for anchorage-independent growth and the metastatic spread of tumour cells. These studies showed that in TC45+TC48, but not TC48, knockdown HeLa cells, SFKs remained hyperactivated when cells were kept in suspension for 30 min (see FIG. 2B; 0 min collagen); this coincided with the hyper-tyrosine phosphorylation (pTyr) of several proteins, in particular that of a protein of ˜70 kDa (FIG. 2B) that co-migrated with cortactin (data not shown), which is an established substrate of c-Src and an oncoprotein implicated in breast cancer cell migration and invasion^{45,46}.

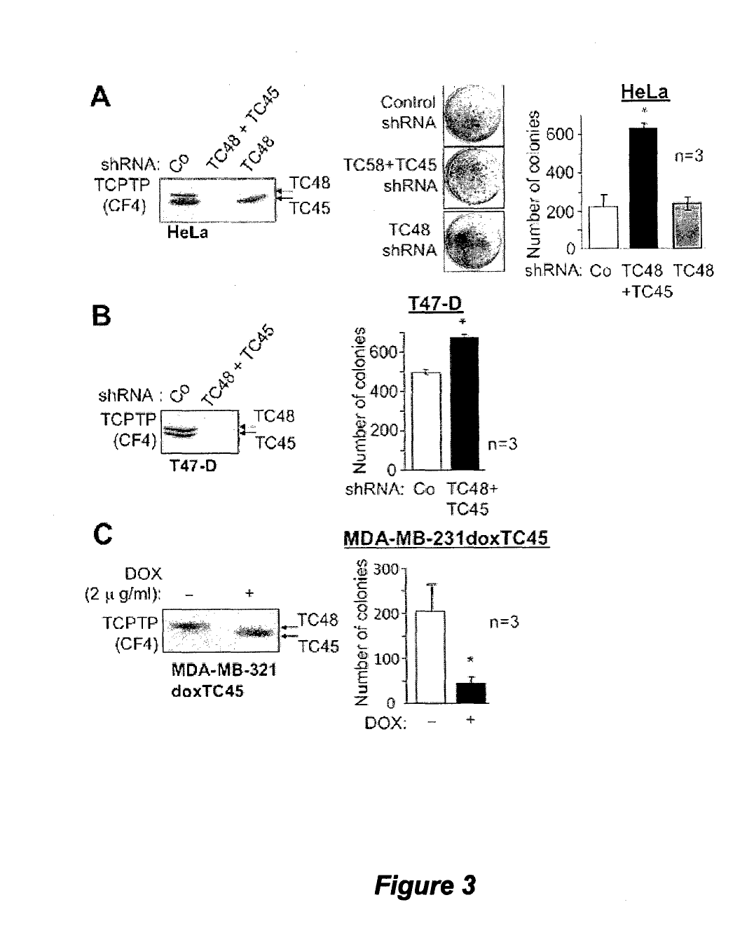

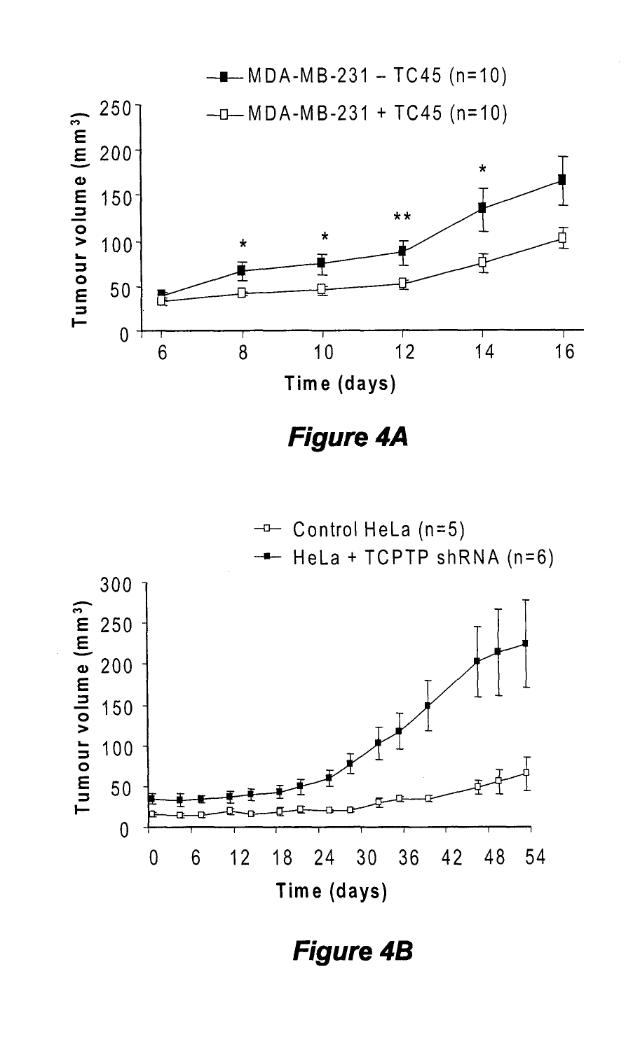

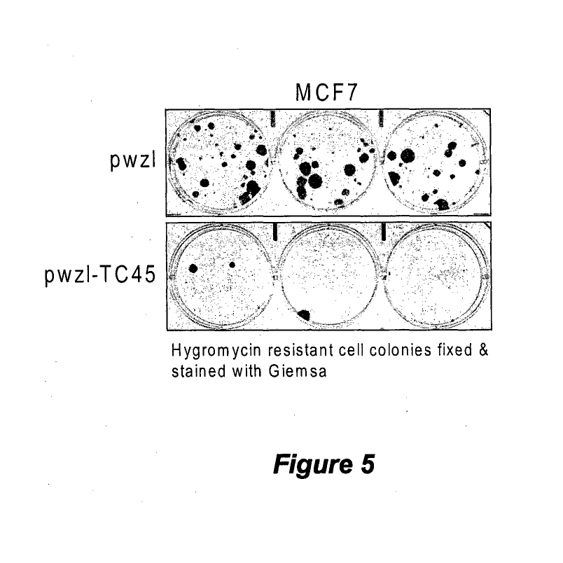

Consistent with the enhanced ligand/integrin-induced activation of PTK pathways and the maintenance of SFK signalling after cell detachment, it was also found that tumorigenicity (as assessed by anchorage-independent growth in soft agar) was significantly enhanced in the TC45+TC48, but not TC48, knockdown HeLa cells (see FIG. 3A). However, while HeLa cells are appropriate for assessing TCPTP's potential to regulate tumorigenicity in general, an investigation was also conducted to assess whether deficiencies in TCPTP expression may affect the tumorigenicity of breast cancer cells. In this regard, when TC45+TC48 were knocked down in ER⁺T47D mammary ductal carcinoma cells (ATCC Accession No HTB-133) by RNAi (see FIG. 3B), and TC45 reconstituted in MDA-MB-231 ErbB1⁺ mammary adenocarcinoma cells (ATCC Accession No HTB-26; normally express TC48 but not TC45) using a doxycycline (DOX)-inducible expression system, and the respective cell growth examined in soft agar (FIG. 3C), it was found that whereas TCPTP knockdown in T47D cells enhanced anchorage-independent growth (FIG. 3B), TC45 reconstitution in MDA-MB-231 cells suppressed growth in soft agar (FIG. 3C). Further, it was found that TC45 expression suppressed the growth of MDA-MB-231 cells in vivo in xenografts implanted in the flanks of Balb/c nude mice (see FIG. 4A). In similar studies, TCPTP knockdown in HeLa cells enhanced the growth of xenografts implanted in the flanks of Balb/c nude mice as determined by tumour volume (FIG. 4B) and tumour weight (data not shown). Therefore, it is proposed that a deficiency in TC45 alone is sufficient to promote/enhance oncogenic PTK signalling and contribute to the tumorigenic process in breast cancer. Significantly, when TC45 was reconstituted into MCF-7 breast cancer cells (that have low TC45 expression; ATCC Accession No HTB-22), it was found that the TC45 inhibited cell proliferation and resulted in cell death (FIG. 5). A similar result was obtained when TC45 was reconstituted in MDA-MB-175 cells (ErbB1/2⁺; ATCC Accession No HTB-25)(data not shown).

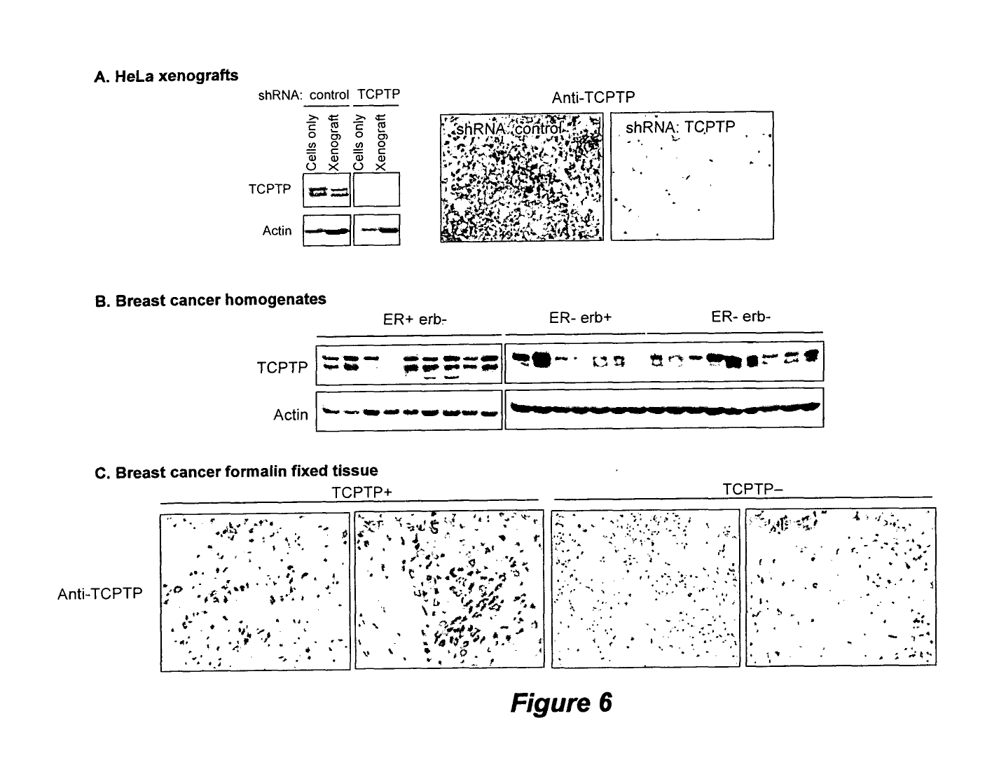

Hypothesising that reduced TCPTP levels in human breast cancer cooperates with ErbB1, SFKs and/or STAT3 to promote tumorigenicity, immunohistochemical (IHC) analysis of PFA fixed HeLa cells was conducted using monoclonal antibody CF4 (Calbiochem Corp, Torrey Pines, Calif., United States of America) that specifically recognises endogenous TCPTP (see the absence of signal in the immunoblots of FIG. 3) after TCPTP knockdown by RNAi, as well as PFA fixed and paraffin embedded sections of human lymph node and infiltrating ductal carcinoma. The results are shown in FIG. 6. It was found that CF4 immunoreactivity was specific for TCPTP in formalin fixed tumour xenografts (FIG. 6A) and produced staining in both breast tissue homogenates (FIG. 6B) and sections (FIG. 6C). Also, CF4 immunoreactivity was observed in the nuclei of lymphocytes in human lymph nodes, consistent with TC45's nuclear location and abundance in the haematopoietic compartment, whereas TCPTP was noted in the nucleus and cytoplasm of epithelioid cells in an infiltrating ductal carcinoma sample (see FIG. 6C), consistent with the expression of TC45 and TC48. By removing the paraffin from these sections, rehydrating to water through descending alcohol, and then processing for IHC, it will be possible to monitor for TCPTP (CF4) nuclear (TC45) v/s cytoplasmic (TC48) staining and correlate this with ErbB1 (pY1173), SFK (pY418) and STAT3 (pY705) phosphorylation.

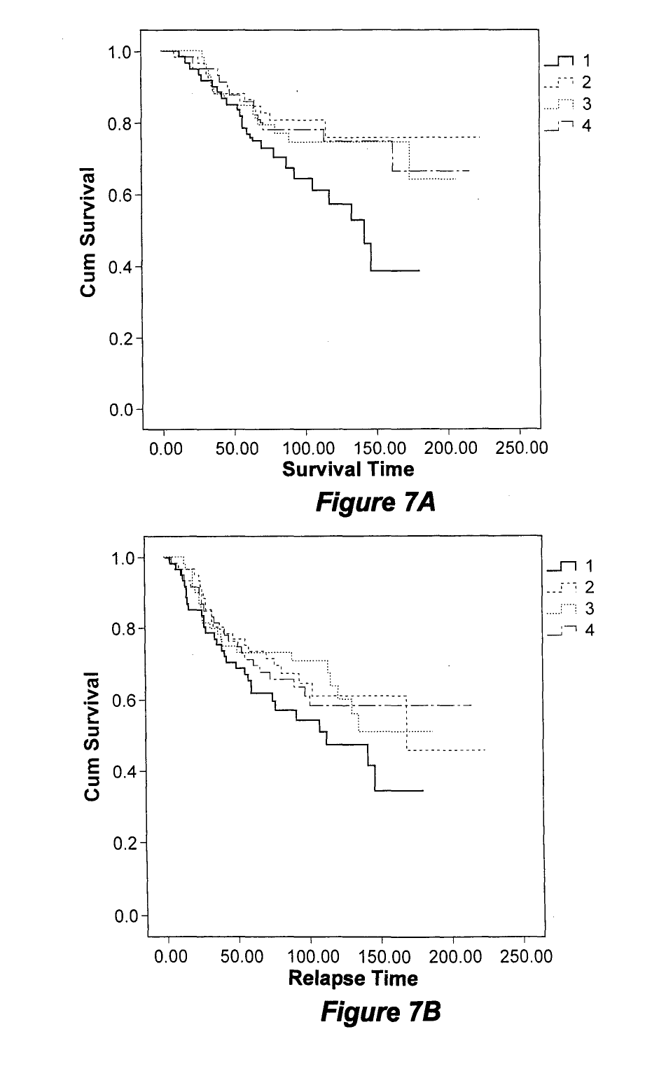

In an interrogation of gene expression data from the tumours of 243 stage I/II breast cancer patients, it was found that Ptpn2 expression was normally-distributed among the 243 tumours. Therefore, for the purposes of further analysis, the patients were split into quartiles of Ptpn2 expression. It was found that patients of the lowest quartile of Ptpn2 expression had a significantly poorer BCSS than the other quartiles (FIG. 7A). Further, the lowest quartile of Ptpn2 expressors had a poorer level of relapse-free survival than the other quartiles (FIG. 7B). Using Chi-square analyses, it was also found that the lowest quartile of Ptpn2 expressors were significantly more likely to have large, ER negative, high grade tumours of basal-like subtype (which is a sub-type associated with poor clinical outcomes and is the subtype observed in BRCA1-related breast cancers⁵¹)(Table 1).

The results of IHC analysis of tumour tissue samples from 119 breast cancer patients are shown in Table 2. These results indicate that TCPTP protein is not detected in 67% of all triple-negative or basal-like tumours that are negative for ER, PR and HER2. Thus, changes in Ptpn2 expression (FIG. 7) correlate with changes in TCPTP protein (Table 1). These results demonstrate that TCPTP status may be used as marker for at least triple-negative or basal-like tumours independent of other variables.

Discussion

The studies of this example have shown that TCPTP protein levels vary in human breast cancer cell lines, that TCPTP-deficiency impacts on tumorigenicity, and that TC45 expression in vivo appears to suppress breast tumour growth (i.e. as observed with MDA-MB-231 tumour (subcutaneous) xenografts). These results strongly indicate that TCPTP deficiency in breast tumour cells may be used as the basis of a diagnostic and/or prognostic assay. To assess this further, additional studies will be conducted to assess TCPTP status in additional breast cancer cell lines with distinct genetic/epigenetic modifications (e.g. MDA-MB-231 (ErbB1⁺; p-SFK and p-STAT3 high), MCF-7 (ER⁺; p-SFK and p-STAT3 low; ATCC Accession No HTB-22) and MDA-MB-175 (ErbB1/2⁺; ATCC Accession No HTB-175) cells reconstituted with TC45, v/s T47D (ER⁺; c-Src⁺; p-SFK high; ATCC Accession No HTB-133) and MDA-MB-436 (ErbB1⁺; ATCC Accession No HTB-130) cells in which TCPTP has been knocked down stably by RNAi or reconstituted by retroviral means). It is anticipated that these additional studies will confirm the utility of TCPTP-deficiency in diagnostic and/or prognostic assays for breast cancer, and additionally, corroborate the finding that TCPTP-deficiency enhances the tumorigenicity of ER+ and ErbB2+ breast cancer cells as well as basal-like triple-negative breast cancer cells by promoting ErbB1, SFK and STAT3 signalling (indeed, in some additional experiments conducted by the present applicant, it was found that IL-6-induced STAT3 signalling is increased in TCPTP (TC45+TC48) knockdown HeLa cells). Further, and while not wishing to be bound by theory, it is predicted that further studies will show that TCPTP loss results in the promotion of PTK signalling and acts cooperatively with c-Src or ErbB1 to promote cellular proliferation, migration/invasion and anchorage-independent growth, thus perturbing the formation of acini in breast epithelium which can lead to the development of ductal carcinoma such as DCIS.

TABLE 1

| | | ER Status | | | Histological Grade | | | | Lymph Node Status | | | |
|---|---|---|---|---|---|---|---|---|---|---|---|---|
| | | Negative | Positive | p-value | 1 | 2 | 3 | p-value | pN0 | pN1 | pN2+ | p-value |
| PTPN2 | 1 | 24 | 37 | <0.001 | 9 | 14 | 38 | 0.004 | 35 | 16 | 10 | 0.406 |
| Quartile | | (39.3%) | (60.7%) | | (14.8%) | (23%) | (62.3%) | | (57.4%) | (26.2%) | (16.4%) | |
| | 2 | 10 | 51 | | 21 | 18 | 22 | | 28 | 28 | 5 | |
| | | (16.4%) | (83.6%) | | (34.4%) | (29.5%) | (36.1%) | | (45.9%) | (45.9%) | (8.2%) | |
| | 3 | 16 | 45 | | 14 | 27 | 20 | | 30 | 24 | 7 | |
| | | (26.2%) | (73.8%) | | (23.0%) | (44.3%) | (32.8%) | | (49.2%) | (39.3%) | (11.5%) | |
| | 4 | 4 | 56 | | 14 | 25 | 21 | | 29 | 22 | 9 | |
| | | (6.7%) | (93.3%) | | (23.3%) | (41.7%) | (35.0%) | | (48.3%) | (36.7%) | (15.0%) | |

| | | Molecular Subtype | | | | | | | Tumour Size | | |
|---|---|---|---|---|---|---|---|---|---|---|---|
| | | Basal- | HER2+/ | Normal- | Normal- | Luminal | Luminal | | <20 | >20 | |
| | | like | ER− | Good | Poor | A | B | p-value | mm | mm | p-value |
| PTPN2 | 1 | 25 | 6 | 0 | 1 | 17 | 12 | <0.001 | 22 | 39 | 0.037 |
| Quartile | | (41.0%) | (9.8%) | (0%) | (1.6%) | (27.9%) | (19.7%) | | (36.1%) | (63.9%) | |
| | 2 | 9 | 8 | 1 | 1 | 26 | 16 | | 38 | 23 | |
| | | (14.8%) | (13.1%) | (1.6%) | (1.6%) | (42.6%) | (26.2%) | | (62.3%) | (37.7%) | |
| | 3 | 8 | 11 | 4 | 4 | 26 | 8 | | 31 | 30 | |
| | | (13.1%) | (18.0%) | (6.6%) | (6.6%) | (42.6%) | (13.1%) | | (50.8%) | (49.2%) | |
| | 4 | 2 | 6 | 5 | 2 | 32 | 13 | | 31 | 29 | |
| | | (3.3%) | (10.0%) | (8.3%) | (3.3%) | (53.3%) | (21.7%) | | (51.7%) | (48.3%) | |

TABLE 2
IHC screening of breast cancer patients

| | Total | TCPTP (+) | TCPTP (−) | |
|---|---|---|---|---|
| | (n = 119) | (n = 85) | (n = 34) | P value |
| Grade | | | | 0.268 |
| 1 or 2 | 75 | 56 (75%) | 19 (25%) | |
| 3 | 36 | 23 (64%) | 13 (36% | |
| Unknown | 8 | 6 (75%) | 2 (25%) | |
| ER | | | | 0.0036 |
| ER-positive | 74 | 60 (81%) | 14 (19%) | |
| ER-negative | 45 | 25 (56%) | 20 (44%) | |
| PR | | | | 0.0418 |
| PR-positive | 64 | 51 (80%) | 13 (20%) | |
| PR-negative | 55 | 34 (62%) | 21 (38%) | |
| HER2 | | | | 0.5526 |
| HER2-positive | 16 | 13 (81%) | 3 (19%) | |
| HER2-negative | 103 | 72 (70%) | 31 (30%) | |
| Receptor status I | | | | <0.0001 |
| ER/PR/HER2 | 95 | 77 (81%) | 18 (19%) | |
| Triple-negative | 24 | 8 (33%) | 16 (67%) | |
| Receptor status II | | | | <0.0001 |
| Luminal A | 79 | 64 (81%) | 15 (19%) | |
| Triple-negative | 24 | 8 (33%) | 16 (67%) | |
| Other | 16 | 13 (81%) | 3 (19%) | |
| | Total | TCPTP (+) | TCPTP (−) | |
| Feature | (n = 24) | (n = 8) | (n = 16) | P value |
| Basal phenotype | | | | 0.6674 |
| 5 negative | 8 | 2 (25%) | 6 (75%) | |
| CBP | 16 | 6 (38%) | 10 (62%) | |

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by the person skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

1. Yeatman, T. J. Nat Rev Cancer 4:470-480 (2004).

2. Yu, H. and Jove, R. Nat Rev Cancer 4:97-105 (2004).

3. Yarden, Y. Oncology 61 (Suppl 2):1-13 (2001).

4. Di Marco, E. et al. Mol Cell Biol 10:3247-3252 (1990).

5. Muller, W. J. et al. Cell 54:105-115 (1988).

6. Schade, B. et al. Cancer Res 67, 7579-7588 (2007).

7. Corkery, B. et al. Ann Oncol 20(5):862-867 (2009).

8. Nielsen, T. O. et al. Clin Cancer Res 10:5367-5374 (2004).

9. Muthuswamy, S. K. et al. Nat Cell Biol 3:785-792 (2001).

10. Biscardi, J. S. et al. J Biol Chem 274:8335-8343 (1999).

11. Olayioye, M. A. et al. Exp Cell Res 267:81-87 (2001).

12. Hsieh, F. C. et al. Biochem Biophys Res Commun 335:292-299 (2005).

13. Diaz, N. et al. Clin Cancer Res 12:20-28 (2006).

14. Wilson, G. R. et al. Br J Cancer 95:1410-1414 (2006).

15. Muthuswamy, S. K. and Muller, W. J. Oncogene 11:1801-1810 (1995).

16. Muthuswamy, S. K. and Muller, W. J. Oncogene 11:271-279 (1995).

17. Finn, R. S. Ann Oncol 19:1379-1386 (2008).

18. Hiscox, S. et al. Breast Cancer Res Treat 115(1):57-67 (2008).

19. Alvarez, J. V. et al. Cancer Res 65:5054-5062 (2005).

20. Garcia, R. et al. Oncogene 20:2499-2513 (2001).

21. Heinrich, P. C. et al. Biochem J374:1-20 (2003).

22. Selander, K. S. et al. Cancer Res 64:6924-6933 (2004).

23. Sansone, P. et al. J Clin Invest 117:3988-4002 (2007).

24. Ling, X. and Arlinghaus, R. B. Cancer Res 65:2532-2536 (2005).

25. Gritsko, T. et al. Clin Cancer Res 12:11-19 (2006).

26. Andersen, J. N. et al. Faseb J 18:8-30 (2004).

27. Zhang, S. Q. et al. Mol Cell 13:341-355 (2004).

28. Zhu, S. et al. Cancer Res 67:10129-10137 (2007).

29. Bentires-Alj, M. and Neel, B. G. Cancer Res 67:2420-2424 (2007).

30. Julien, S. G. et al. Nat Genet 39:338-346 (2007).

31. Tonks, N. K. and Muthuswamy, S. K. Cancer Cell 11:214-216 (2007).

32. Ruivenkamp, C. A. et al. Nat Genet 31:295-300. (2002).

33. Ruivenkamp, C. et al. Oncogene 22:3472-3474 (2003).

34. Motiwala, T. et al. Proc Natl Acad Sci USA 101:13844-13849 (2004).

35. Wang, Z. et al. Science 304:1164-1166 (2004).

36. Lam, M. H. et al. J Biol Chem 276:37700-37707 (2001).

37. Galic, S. et al. Mol Cell Biol 23:2096-2108 (2003).

38. Simoncic, P. D. et al. Curr Biol 12:446-453 (2002).

39. ten Hoeve, J. et al. Mol Cell Biol 22:5662-5668 (2002).

40. Aoki, N. and Matsuda, T. Mol Endocrinol 16:58-69 (2002).

41. Yamamoto, T. et al. Biochem Biophys Res Commun 297:811-817 (2002).

42. Klingler-Hoffmann, M. et al. J Biol Chem 276:46313-46318. (2001).

43. Mattila, E. et al. Nat Cell Biol 7:78-85 (2005).

44. WTCCC Nature 447:661-678 (2007).

45. Timpson, P. et al. Cancer Res 67:9304-9314 (2007).

46. Clark, E. S. et al. Cancer Res 67:4227-4235 (2007).

47. Wojdacz, T. K. and Dobrovic, A. Nucleic Acids Res 35:e41 (2007).

48. Nguyen, C. et al. J Natl Cancer Inst 93:1465-1472 (2001).

49. van de Vijver M. J. et al. N Engl J Med 347:1999-2009 (2002).

50. Glas A. M. et al. BMC Genomics 7:278 (2006).

51. Livasy C. A. et al. Modern Pathology 19:264-271 (2006).

What is claimed is:

1. A method of diagnosis and/or prognosis of cancer in a subject, said method comprising the step of assessing, in a suitable body sample from said subject, any lack or reduction of TCPTP protein and/or Ptpn2 gene expression.

2. A method for assessing a subject's predisposition to cancer, said method comprising the step of assessing, in a suitable body sample from said subject, any lack or reduction of TCPTP protein and/or Ptpn2 gene expression.

3. A method for assisting the selection of a therapy for a cancer in a subject, said method comprising the step of assessing, in a suitable body sample from said subject, any lack or reduction of TCPTP protein and/or Ptpn2 gene expression.

4. The method of any one of claims 1 to 3, wherein the method comprises:
(i) a first step of assessing, in a first suitable body sample from said subject, any lack or reduction of TCPTP protein and/or Ptpn2 gene expression; and
(ii) one or more further step(s) of assessing any lack or reduction of TCPTP protein and/or Ptpn2 gene expression, in a body sample(s) that is substantially equivalent to the first body sample but taken from the subject at one or more later time point(s).

5. The method of claim 4, wherein the method comprises detecting the amount of TC45 protein.

6. The method of any claim 5, wherein the cancer is a breast cancer selected from the group consisting of those that are ER positive ErbB2 negative (ER⁺ErbB2⁻), ER negative ErbB2 positive (ER⁻ErbB2⁺) and ER negative ErbB2 negative (ER⁻ErbB2⁻).

7. The method of claim 5, wherein the cancer is a breast cancer that is a triple-negative tumour (i.e. ER negative ErbB2 negative progesterone receptor (PR) negative; ER⁻ErbB2PR⁻).

8. The method of claim 5, wherein the body sample(s) is a tumour tissue sample.

9. A method of treating or preventing cancer in a subject, wherein said method comprises administering to said subject an agent for modulating the activity of TCPTP, optionally in combination with a pharmaceutically acceptable carrier and/or excipient.

10. The method of claim 9, wherein the agent is selected from the group consisting of TC45, TC48, agents which enhance transcription or translation of the Ptpn2 gene, gene therapy agents containing a polynucleotide sequence encoding TC45 or TC48, functional fragments of TCPTP, peptide mimetics of the active domains of TCPTP, and small organic molecules which mimic TCPTP activity.

11. (canceled)

12. (canceled)

13. (canceled)

14. A pharmaceutical composition for treating or preventing cancer, said composition comprising an agent for modulating the activity of TCPTP in combination with a pharmaceutically acceptable carrier and/or excipient.

15. The composition of claim 14, wherein the agent is selected from the group consisting of TC45, TC48, agents which enhance transcription or translation of the Ptpn2 gene, gene therapy agents containing a polynucleotide sequence encoding TC45 or TC48, functional fragments of TCPTP, peptide mimetics of the active domains of TCPTP, and small organic molecules which mimic TCPTP activity.

16. A kit for use in the method of claim 1, wherein said kit comprises a TCPTP protein or a functional fragment thereof and/or an anti-TCPTP antibody or fragment thereof.
